(19) United States
(12) Patent Application Publication
(10) Pub. No.: US 2022/0233134 A1
(43) Pub. Date: Jul. 28, 2022

(54) System and Method for Stroke Detection and Prevention

(71) Applicant: Xiaoling Zhao, Irvine, CA (US)

(72) Inventor: Xiaoling Zhao, Irvine, CA (US)

(21) Appl. No.: 17/718,064

(22) Filed: Apr. 11, 2022

Related U.S. Application Data
(63) Continuation of application No. PCT/US2020/055502, filed on Oct. 14, 2020.

(51) Int. Cl. A61B 5/00; A61B 5/242

(52) U.S. Cl. CPC A61B 5/4064; A61B 2562/0223; A61B 5/6802; A61B 5/242

(57) ABSTRACT

A method and a system for detecting the possible existence of a stroke is provided. In a first step of the method, a first measurement of a nerve impulse is obtained from the skin of a first location of a patient using a magnetometer. In a second step, the first measurement is compared with either a baseline measurement or a second measurement of a nerve impulse obtained from the skin of a second location of the patient using a magnetometer. In a third step, a signal is transmitted indicating a possible stroke if there is no symmetry between the first measurement and either the baseline measurement or the second measurement.

### BACKGROUND OF THE INVENTION

### 1. Field of the Invention

The present invention relates to the early detection of stroke, and in particular, to a method and a system for early stroke detection and prevention.

### 2. Description of the Prior Art

According to World Health Organization (WHO), 15 million people suffer from stroke worldwide each year. Of these, 5 million die and another 5 million become permanently disabled. Stroke is the third leading cause of death in the United States, and is the leading cause of serious, long term disability.

Fortunately, stroke is largely preventable. The risk of stroke can be reduced by living a healthy lifestyle, such as by controlling high blood pressure, not smoking, eating a healthy diet, being physically active, maintaining a healthy body weight, managing diabetes, etc. In addition, stroke is treatable. If someone is having a stroke, time is critical. Immediate treatment may minimize the long-term effects of a stroke and even prevent death, and also improve the clinical outcome post treatment. For example, for a patient experiencing ischemic stroke, if the patient can reach a hospital within 3.0 to 4.5 hours after the onset of symptoms, then a clot-dissolving drug called IV Alteplase (tPA) can be administered for treatment. The sooner the drug is administered, the greater the possibility of a better outcome. If the patient reaches the hospital beyond 4.5 hours, but still within 6 to 24 hours, of the onset of the first symptoms, then stroke can be treated by a treatment option known as mechanical thrombectomy.

Unfortunately, patients typically do not recognize the signs of a stroke until it becomes really serious. Thus, although the symptoms of stroke may be well known, many people do not seek treatment until the situation becomes serious due to reasons such as cost, laziness, overconfidence about their own health, busy schedules, or some other excuse.

Therefore, there is a critical need to provide a solution for early detection and prevention of the stroke.

### SUMMARY OF THE DISCLOSURE

The present invention discloses systems and related methods to help detect stroke, and to help patients obtain medical treatment more quickly, for better clinical outcomes.

In order to accomplish the objects of the present invention, there is provided a method of detecting the possible existence of a stroke. In a first step of the method, a first measurement of a nerve impulse is obtained from the skin of a first location of a patient using a magnetometer. In a second step, the first measurement is compared with either a baseline measurement or a second measurement of a nerve impulse obtained from the skin of a second location of the patient using a magnetometer. In a third step, a signal is transmitted indicating a possible stroke if there is a significant difference from baseline or no symmetry between the first measurement and either the baseline measurement or the second measurement.

According to another embodiment of the present invention, the method of detecting the possible existence of a stroke can include the steps of obtaining a first image of a first location of a patient using an optical image capturing device, comparing the first image with either a baseline image or a second image obtained from a second location of the patient using an optical image capturing device, and transmitting a signal indicating a possible stroke if there is a significant difference from the baseline or no symmetry between the first image and either the baseline image or the second image.

### BRIEF DESCRIPTION OF THE DRAWINGS

FIG. 1 is a schematic diagram illustrating one embodiment of a system for stroke detection according to the present invention.

FIG. 2 is a schematic diagram illustrating another embodiment of a system for stroke detection according to the present invention.

FIG. 3 is a schematic diagram illustrating a further embodiment of a system for stroke detection according to the present invention.

FIG. 4 is a schematic diagram illustrating yet another embodiment of a system for stroke detection according to the present invention.

FIG. 5 is a schematic diagram of an atomic magnetometer that can be used with the present invention.

FIG. 6 is a schematic diagram illustrating yet another embodiment of a system for stroke detection according to the present invention.

FIG. 7 is a flowchart illustrating a general method according to the present invention.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

The present invention provides a system and method for stroke detection and prevention through the non-invasive detection of nerve impulse with an atomic magnetometer or optic atomic magnetometer using a probe or sensor near the skin surface of a patient, or to contact the skin surface of the patient. The skin surface includes any skin surface, such as the tongue, nose, nose tip, face, head surface, arm, hand, fingers, and leg, etc. The sensor or probe can be attached on or near the skin surface, either on one side, or both sides, of the body parts mentioned above. For example, the probe can be attached to or placed near the left side of the nose or nose tip first, to detect the nerve impulse, and then the sensor or probe can be attached to, or placed near, the right side of the nose or nose tip to detect the nerve impulse. The results from the left and right sides can either be used independently for stroke detection, or can be used at the same time for comparison, to detect the existence of a stroke that might be occurring, since if a stroke happens in one side of the brain, the nerve impulse can be different from the other side of the body or different from the baseline nerve impulse on the same side. This example can be applied to other body parts mentioned above for stroke detection as well.

Magnetic field measurements allow for the true measurement of the axon's axial net current, which is the depolarizing wavefront driving the action potential (nerve impulse). Magnetic field recordings also make it possible for non-invasive measurement of the conduction velocity of the nerves, which can be used to detect/diagnose abnormities in the nerve system, and hence to detect stroke or other diseases.

In this regard, the human brain and nervous system generate magnetic fields. The magnetic fields are detectable by magnetometers as action potentials (nerve impulse) from the nerve. Atomic magnetometers or optical atomic magnetometers provide the sensitivity needed to detect human nerve impulse through a sensor or probe attached to or near the human skin. By positioning the sensor or probe on or near the skin, with the nerve being under the skin, the magnetic field generated by the action potential (nerve impulse) can be detected and recorded by the magnetometer. The measurements of the magnetic field can be used to determine the activity of the nerve and the temporal shape of the nerve impulse. The magnetic field measurement from one side of the body can either be used independently (compare with the baseline signal from the same side) for stroke detection, or it can be used in combination with the measurement from the opposite side of the body to compare and detect any abnormities that might indicate the presence of a stroke or other disease. In other words, the present invention is based on both detecting the nerve impulse differences in the affected and non-affected sides of the body when a stroke happens, and detecting the stroke by comparing the signal with a baseline signal from the same side.

The use of nerve impulse, and concepts of neural communication, are explained in greater detail in the article “Thinking about the nerve impulse: A critical analysis of the electricity-centered conception of nerve excitability”, by Drukarch et al., Progress in Neurobiology 169 (2018), pages 172-185.

The measurements of the magnetic field can be used to determine the activity of the nerve and the temporal shape of the nerve impulse. For example, a sensor or detection device (e.g., a ring or a device having other shapes/geometries) can be worn on the finger of a person to detect the activity of the nerve and the temporal shape of the nerve impulse. If no nerve impulse (or a weak impulse signal) is detected, then stroke is detected. Also, the device can be worn on a finger in one hand, or fingers in both hands, and if the difference in nerve impulse is detected between two hands, then a stroke is detected. Of course, the ring-like or other shaped detection device can either detect the nerve impulse and provide feedback by itself directly through a sound signal, a visual signal, lights, vibrations, a displayed reading of the results, etc., or it can also be connected with some other device, such as a smart phone, through some APP, platform or software, to communicate a warning or other signal that a possible stroke has been detected.

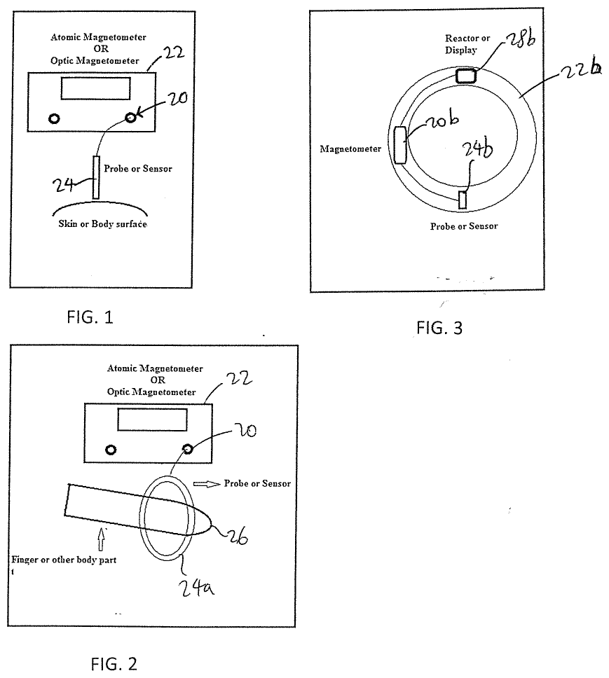

Referring to FIG. 1, the magnetometer 20 can be embodied in the form of a chip that is embedded to, or secured to, a hand-held carrying device 22 (e.g., a keychain, a wristwatch-like device, or similar devices that are worn on a body part, etc.) that also includes a probe that includes a sensor 24 electronically coupled to the magnetometer 20 and the carrying device 22. In FIG. 1, the numeral 22 designates a housing or object which embodies the components of the magnetometer 20 shown in FIG. 5.

Referring to FIG. 5, a conventional atomic magnetometer typically includes three main components: a resonant light source 50, an alkali vapor cell 52, and a sensor 24 that monitors the intensity of the light transmitted through the vapor cell 52. In one embodiment, the light source 50 can be a laser. Also, the sensor 24 can be a photodetector, photodiode, or any other detection mechanism.

One example of an atomic magnetometer that can be used for biomedical applications is found in “A Compact, High Performance Atomic magnetometer for Biomedical Applications”, by Shah & Wakai, Phys Med Biol. 2013 Nov. 21. In addition, the basic operating principles behind a conventional magnetometer are described in “Non-invasive detection of animal nerve impulses with an atomic magnetometer operating near quantum limited sensitivity”, by Jensen et al., Scientific Reports, Jan. 2016, | 6:29638 | DOI: 10.1038/srep29638. These two references are incorporated by this reference as though set forth fully herein.

The magnetometer 20 can also be an optical magnetometer. Optical magnetometry makes use of various optical techniques to measure magnetization.

If desired, a signal magnifier and/or a filter can be added into the magnetometer 20 to enhance the signal.

In use, the probe can be held against the surface of a person's skin, as shown in FIG. 1, and the sensor 24 detects the nerve impulse which is used to calculate the magnetic field using techniques that are well known in the art, including those described above.

The magnetometer 20 can be incorporated in a wearable device. FIG. 2 shows a ring 24a being used as a sensor. The patient wears the ring 24a around his or her finger 26 and the sensor 24 on the ring 24a detects the nerve impulse.

FIG. 3 illustrates a ring being used as the carrying device 22a. The magnetometer 20b and the sensor 24b are both incorporated in the ring, and are electronically coupled to each other, and to a reactor or display 28b. The reactor or display 28b can be a display that provides a signal or other indication that a stroke is being detected. For example, the signal can be a flashing red light which indicates that a stroke is being detected, while a constant non-flashing light signal can be used to indicate that no stroke is detected. The ring 22b can be worn on fingers on both hands in order to generate the desired signal.

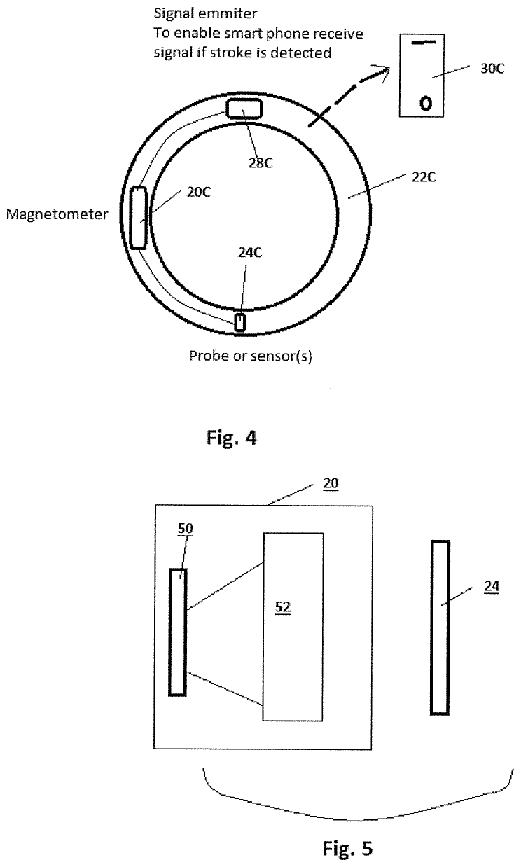

FIG. 4 illustrates the same system as shown in FIG. 3, except that the display 28b is replaced by a transmitter or emitter 28c. The magnetometer 20c and the sensor 24c are both incorporated in the ring, and are electronically coupled to each other, and to the emitter 28c. The emitter 28c is adapted to emit or transmit a signal to a smart device 30c (e.g., smart phone) or a computer which will display the results detected by the sensor 24c and calculated by the magnetometer 20c. The presence or absence of a stroke will be displayed or indicated at the smart device 30c or the computer. For example, the smart phone can provide immediate feedback to the user on the nerve impulse measurement results, so that the patient can receive the warning immediately if a stroke is detected, and prompt the user to seek medical attention immediately.

The system and method of the present invention can be adapted for use in a variety of applications.

For example, an atomic magnetometer can also be used in connection with the eyes of the human. A sensor 24 can be placed or positioned on the skin adjacent the upper eyelid, lower eyelid, eyeball, retina, etc. for stroke detection.

As a modification to the embodiment of FIG. 4, the signal from the emitter 28c can be directly sent to a hospital or emergency service through the internet when a stroke is detected.

The carrying device 22 can also be made as a wearable device that can be used either in a hospital setting, a care facility setting, or at home.

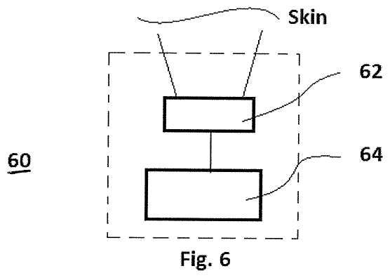

The present invention also provides systems for optical detection of stroke. For example, referring to FIG. 6, a device 60 resembling an optical microscope, which contains a magnifier lens 62 and an image analysis system 64, can be used to detect stroke. Such an optical microscope would observe and capture images of the nose or nose tip from a frontal angle, analyze the geometry symmetry of the left and right sides of the nose using an image analysis software, and if differences are detected (e.g., the two sides have a different geometry or appearance), then a stroke is detected. The device 60 can be embodied in the form of a smart device (e.g., a smart phone or tablet), with the processor acting as the system 64 and having software for analyzing the captured images. The image analysis would analyze the geometry for symmetry using image analysis and comparison techniques that are well-known in the art. For example, if the geometry of the two eyelids (or two sides of the nose, or eyeball, or pupil of the eye, etc.) is not symmetrical, then a possible stroke is detected.

In this regard, the camera on a smart phone can also be used to capture images of the nose or nose tip from a frontal angle, analyze the geometry symmetry of the left and right sides of the nose using an image analysis software, and if differences are detected (e.g., the two sides have a different geometry or appearance), then a possible stroke is detected.

In addition, retinal cameras can be used to collect the geometry information and/or the optical information of a patient's eyeball and/or retina for stroke detection. Using a similar method, the geometry comparison between the left and right eyeball or retina can be used the detect any asymmetry between the two sides for stroke detection. The scanned image of the retina can be analyzed by software for stroke detection as well.

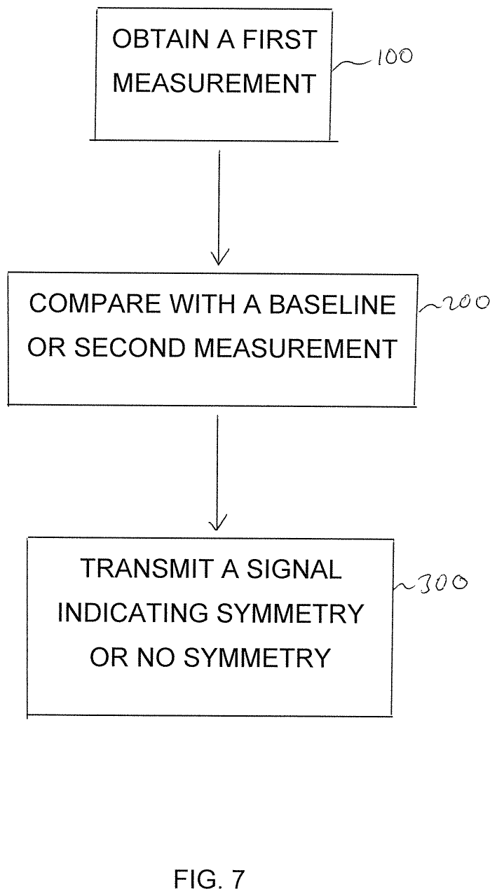

FIG. 7 illustrates a general method or process for the present invention. In the first step 100, a first measurement is obtained. As explained above, this first measurement can be a measurement of a nerve impulse obtained from a magnetometer, or an image captured by an image capturing device, of a desired location of a patient's body. In the second step 200, the first measurement is compared with either a baseline or a second measurement. The baseline can be a known baseline measurement that has been obtained when the system of the present invention is initially set up for use with the specific patient. Alternatively, the second measurement can be a measurement obtained from another part of the patient's body (e.g., the opposite hand or eye) for comparison. This comparison with either a baseline or a second measurement will indicate whether a symmetry exists, or if there is a significant difference with the initially-measured baseline. When a baseline is used for comparison, the symmetry will exist if the first measurement is substantially the same as the baseline. When a second measurement is used for comparison, the symmetry will exist if the first measurement is substantially the same as the second measurement. In the third step 300, a signal is transmitted to indicate whether symmetry exists (i.e., no stroke), or symmetry does not exist (i.e., possible stroke). This signal can be emitted and processed using any of the channels described above, including sound signals, light signals, signals transmitted to a smart device or directly to a clinic or hospital, etc.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A method of detecting the possible existence of a stroke, comprising the steps of:
obtaining a first measurement of a nerve impulse from the skin of a first location of a patient using a magnetometer;
comparing the first measurement with either a baseline measurement or a second measurement of a nerve impulse obtained from the skin of a second location of the patient using a magnetometer; and
transmitting a signal indicating a possible stroke if there is no symmetry between the first measurement and either the baseline measurement or the second measurement.

2. The method of claim 1, wherein the step of obtaining uses an atomic magnetometer.

3. The method of claim 1, wherein the first location is on the left side of the patient's body and the second location is on the right side of the patient's body.

4. The method of claim 2, wherein the atomic magnetometer includes a light source, an alkali vapor cell, and a sensor.

5. The method of claim 1, wherein the step of transmitting includes the step of transmitting the signal to a smart device.

6. The method of claim 4, wherein the magnetometer is incorporated in a wearable device that is worn by the patient.

7. A method of detecting the possible existence of a stroke, comprising the steps of:
obtaining a first image of a first location of a patient using an optical image capturing device;
comparing the first image with either a baseline image or a second image obtained from a second location of the patient using an optical image capturing device; and
transmitting a signal indicating a possible stroke if there is no symmetry between the first image and either the baseline image or the second image.

8. The method of claim 7, wherein the first location is on the left side of the patient's body and the second location is on the right side of the patient's body.

9. The method of claim 7, wherein the step of transmitting includes the step of transmitting the signal to a smart device.
